(19) United States
(12) Patent Application Publication
(10) Pub. No.: US 2007/0179334 A1
(43) Pub. Date: Aug. 2, 2007

(54) SLEEP AID

(76) Inventors: Laurie Leslie GROVES, Aptos, CA (US); Michael W. STARKWEATHER, Sandy, UT (US)

Correspondence Address: ADVANTIA LAW GROUP, 9035 SOUTH 1300 EAST, SUITE 200, SANDY, UT, 84094, US

(21) Appl. No.: 11/307,274

(22) Filed: Jan. 30, 2006

(51) Int. Cl. A61M 21/00; G09B 23/28

(52) U.S. Cl. 600/28; 434/266; 600/26

(57) ABSTRACT

A sleep aid device for inducing and/or maintaining sleep which includes a fluid-filled mattress, a fluid pump sealably connected to the mattress, and a control module. The fluid pump modulates fluid into and/or out of the mattress, which may simulate the feel and/or sound of a human breathing. Also, the sleep aid device may include a heartbeat module which simulates the feel and/or sound of a human heartbeat. Further disclosed is a method of inducing and/or maintaining sleep by simulating the sound and/or feel of breathing and/or heartbeat, and controlling the rates of the simulated breathing and/or heartbeat to induce and/or maintain sleep.

### FIELD OF THE INVENTION

The present invention relates to sleep aids, specifically to devices for simulates a human breath.

### DESCRIPTION OF THE RELATED ART

Humans need sleep. Normally, sleep and wakefulness occur in a cyclical manner, typically integrated with the 24-hour solar day. These daily cyclic patterns are known as circadian rhythms. Generally, adults need about 8 hours of sleep per day, depending on various factors. Teenagers need about 9 hours of sleep per day. Infants need from about 16 to 18 hours of sleep per day. Typically, people pass through five stages repeatedly during sleep, the deepest of which is known as Rapid Eye Movement (REM). The first four stages of sleep are known as non-REM sleep. During the first stage there is about a 50% reduction in activity between wakefulness and sleep. This stage typically lasts about 10 minutes. During stage 2, the body temperature decreases, the heart rate decreases, and the muscles pass through phases of tension and relaxation. Stages 3 and 4 are deep sleep. During these non-REM stages of sleep, the body typically performs regenerative tasks such as repairing tissues, building bone and muscle, and strengthening the immune system.

REM sleep typically occurs after about 90 minutes of non-REM sleep.

Cerebral activity increases during REM sleep, and people may experience intense dreaming during REM sleep. People with sleep disorders, however, may also experience erratic increases in heart rate and breathing rate during REM sleep. These erratic increases at times cause the person to awake or pass back into non-REM sleep.

Breathing rate changes during sleep. Breathing typically slows during the first two stages of non-REM sleep, when a person may experience brief periods (5-15 seconds) of not breathing, a pattern known as periodic breathing. Breathing stabilizes during stages 3 and 4 of non-REM sleep. During these stages breathing rate is typically slower than during wakefulness. A person may breathe about 1 to 2 L/minute less than during wakefulness. During REM sleep, breathing periods are less regular, and may include brief periods of not breathing. However, the breathing during REM sleep is not periodic.

Infants, children and adults often experience difficulty in sleeping. One cause of this difficulty is the infant, child or adult may not be able to control his/her breathing such that it slows sufficiently to achieve sleep, or achieve a deep sleep. Particularly, infants and children who are born to mothers who used drugs, especially stimulants, while the infant was in the womb have difficulty in controlling breathing and achieving sleep or deep sleep.

In U.S. Pat. No. 4,934,997, Skakas discloses a therapeutic infant bed primarily intended for hospital use in the treatment and prevention of infant apnea. The bed comprises a hammock-forming sheet of material having one surface thereof covered with a soft tactile material. The hammock-forming sheet of material includes means for attaching a sonic device for generating audible sounds and mechanical vibrational impulses simulating a human heartbeat thereto and for causing the audible sounds and mechanical vibrational impulses to be transmitted through the sheet and the support structure to an infant disposed in the bed. A support structure is provided which has raised, spaced end members defining an open space therebetween. The hammock-forming sheet of material is horizontally suspended between the end members with the one surface facing up between the end members, within the open space, and above a surface upon which the support structure is sitting to form a hammock having spaced head and foot ends and spaced sides between which an infant can be placed. Rocking means are operably attached to the support structure for supporting the support structure on a planar surface and for slowly and gently rocking the support structure primarily only in a horizontal plane. In the preferred embodiment, the rocking means comprises a plurality of spring suspension members self activated by the infant's movements and disposed under the support structure.

Further, Blitzer discloses, in U.S. Pat. No. 4,947,832 that a colicky infant's vestibular and auditory centers are stimulated mechanically to simulate womb conditions to ease the transition from womb to postgestational life. In this soothing method an infant is placed in a crib with a mattress and having padded protective sides assuring appropriate positioning on the mattress, to which is imparted a regular, repetitive low frequency sine wave motion progressively traveling longitudinally thereof with simultaneous vibrations, whereby the infant experiences gentle, low frequency, regular, progressive, traveling sine wave motion plus hearing and sensing subdued vibrations like sensations previously experienced in the womb. The protective padded sides encircling the infant offer a warm enclosure while subduing or muffling exterior sounds. The illustrative apparatus includes an upper, mattress-supporting layer having a longitudinal series of transverse slats, gently moved up and down by motor-driven cams providing a distributed drive producing regular, repetitive, progressively traveling sine wave motion simulating peristalsis effects of an intenstine near a uterine wall. This support layer is also driven by vibrators simulating inter-uterine pulsations of a mother's blood, heartbeat, body movements, etc. A one-inch thick foam mattress covering the slats provides the longitudinally traveling sine wave motion undulating up and down about 2 to 7 millimeters (preferably 3.5 to 6.5 mm) at about 7 to 20 cycles per minutes, optimally about 8 to 12 cycles per minute, with wave length from about 24 inches to about 3 inches, and presently preferred from about 18 inches to about 4 inches.

Cummins discloses, in U.S. Pat. No. 4,066,072, comfort cushion for infants comprising in combination a fluid-filled flexible elastic hermetically sealed infant supporting mattress; a pulsating fluid pump means having mattress duct and outlet fluid conducting conduits communicating between the pump means and the mattress for circulating a substantially non-compressible fluid through the mattress; and an infant heart beat and breathing alarm means having sensor means embedded in a mattress infant supporting surface for actuating an alarm means external to the mattress if an infant supported on the mattress were to stop breathing or its heart were to stop beating or if either breathing or heart beat were to change such as to indicate peril to the infant. The mattress duct conduit has a pressure activated valve within the mattress which opens at a pre-selected fluid flow pressure and closes upon the pressure dropping below a lower pre-selected pressure to simulate sounds and fluid movement conditions a pre-born infant experiences.

What is needed is a sleep aid that solves one or more of the problems described herein and/or one or more problems that may come to the attention of one skilled in the art upon becoming familiar with this specification.

### SUMMARY OF THE INVENTION

The present invention has been developed in response to the present state of the art, and in particular, in response to the problems and needs in the art that have not yet been fully solved by currently available sleep aids. According to one embodiment of the present invention there is a sleep aid for assisting the modification of a breathing rate in a human, comprising: a fluid-filled, and substantially flexible mattress which includes a fluid duct; a fluid pump sealably connected to a conduit, which is sealably connected to the fluid duct, and wherein the fluid pump modulates the fluid into the mattress at a modulate rate; and a control module, which controls the modulate rate of the fluid pump.

The sleep aid may further include a detector module which receives signals corresponding to a physiological event of a human, and wherein the control module receives said signals corresponding with a physiological event of a human. The control module may further include a timer module. The control module may cause the modulate rate to decrease stepwise over time to a predetermined modulate rate. The control module may cause the modulate rate to decrease continuously over time to a predetermined modulate rate. The control module may decrease the modulate rate as a function of time as determined by the timer module. The control module may cause the modulate rate to decrease and increase in accordance with a pattern. The pattern may correspond to a breathing rate pattern of a sleeping adult human. The control module may cause the modulate rate to decrease and increase corresponding with the signals received by the control module from the detector module. The sleep aid may further include a breathing module that simulates the sound of breathing when a fluid passes therethrough. The sleep aid may further include a heartbeat device coupled to the control module that simulates the sound of a heartbeat. The heartbeat device may simulate the sound of a heartbeat at a rate controlled by the control module. The rate of the heartbeat may increase and/or decrease with the modulate rate.

According to another embodiment of the present invention there is a method of assisting the modification of a breathing rate in a human, comprising the steps of: placing the human on a fluid-filled and substantially flexible mattress including a fluid duct, which is sealably connected to a fluid pump which modulates the fluid into the mattress at a modulate rate and includes a control module, which controls the modulate rate of the fluid pump; modulating the fluid into the mattress at a modulate rate; and decreasing.

The step of decreasing the modulate rate may correspond to a sleep pattern of the human. The method may further include providing a sound simulating a heartbeat of a human. The method may further include increasing the modulate rate. The decreasing and increasing the modulate rate may correspond to breathing rate pattern of a sleeping human. The method may further include the step of providing a sound which simulates breathing of a human. The sound simulating breathing of a human may repeat at a rate corresponding to the rate of modulating the fluid into the mattress.

Reference throughout this specification to features, advantages, or similar language does not imply that all of the features and advantages that may be realized with the present invention should be or are in any single embodiment of the invention. Rather, language referring to the features and advantages is understood to mean that a specific feature, advantage, or characteristic described in connection with an embodiment is included in at least one embodiment of the present invention. Thus, discussion of the features and advantages, and similar language, throughout this specification may, but do not necessarily, refer to the same embodiment.

Furthermore, the described features, advantages, and characteristics of the invention may be combined in any suitable manner in one or more embodiments. One skilled in the relevant art will recognize that the invention can be practiced without one or more of the specific features or advantages of a particular embodiment. In other instances, additional features and advantages may be recognized in certain embodiments that may not be present in all embodiments of the invention.

These features and advantages of the present invention will become more fully apparent from the following description and appended claims, or may be learned by the practice of the invention as set forth hereinafter.

### BRIEF DESCRIPTION OF THE DRAWINGS

In order for the advantages of the invention to be readily understood, a more particular description of the invention briefly described above will be rendered by reference to specific embodiments that are illustrated in the appended drawings. Understanding that these drawings depict only typical embodiments of the invention and are not therefore to be considered to be limiting of its scope, the invention will be described and explained with additional specificity and detail through the use of the accompanying drawings, in which:

FIG. 1 illustrates a perspective view of the sleep aid according to one embodiment of the present invention;

FIG. 2 illustrates a graph depicting a modulate rate pattern according to one embodiment of the present invention;

FIG. 3 illustrates cross-sectional view of a valve according to one embodiment of the present invention; and

FIG. 4 illustrates a perspective view of the sleep aid according to one embodiment of the present invention.

### DETAILED DESCRIPTION OF THE INVENTION

For the purposes of promoting an understanding of the principles of the invention, reference will now be made to the exemplary embodiments illustrated in the drawings, and specific language will be used to describe the same. It will nevertheless be understood that no limitation of the scope of the invention is thereby intended. Any alterations and further modifications of the inventive features illustrated herein, and any additional applications of the principles of the invention as illustrated herein, which would occur to one skilled in the relevant art and having possession of this disclosure, are to be considered within the scope of the invention.

Reference throughout this specification to “one embodiment,” “an embodiment,” or similar language means that a particular feature, structure, or characteristic described in connection with the embodiment is included in at least one embodiment of the present invention. Thus, appearances of the phrases “one embodiment,” “an embodiment,” and similar language throughout this specification may, but do not necessarily, all refer to the same embodiment, different embodiments, or component parts of the same or different illustrated invention. Additionally, reference to the wording “an embodiment,” or the like, for two or more features, elements, etc. does not mean that the features are related, dissimilar, the same, etc. The use of the term “an embodiment,” or similar wording, is merely a convenient phrase to indicate optional features, which may or may not be part of the invention as claimed.

As used herein, “mattress” includes an item on which a person may sleep. The mattress may be longer or shorter than the person. The mattress may be thick or thin in relation to the person. The mattress may be filled with a substance such as, for example, a fluid, fibers, particles, springs, and the like. The mattress may include a cover. The mattress may be flexible.

As used herein, “modulate” is meant to indicate a change in a rate. For example, in at least one embodiment, a fluid is modulated into a mattress. This is meant to mean that the flow rate of the fluid into and/or out of the mattress is changed. A fluid may be modulated into and/or out of a mattress by forcing fluid into the mattress followed by forcing the fluid out of the mattress. The force may be applied by, for example, a pump, a pressure gradient, and so forth.

Each statement of an embodiment is to be considered independent of any other statement of an embodiment despite any use of similar or identical language characterizing each embodiment. Therefore, where one embodiment is identified as “another embodiment,” the identified embodiment is independent of any other embodiments characterized by the language “another embodiment.” The independent embodiments are considered to be able to be combined in whole or in part one with another as the claims and/or art may direct, either directly or indirectly, implicitly or explicitly.

Functional units described in this specification may be labeled as modules, in order to more particularly emphasize their implementation independence. For example, a module may be implemented as a hardware circuit comprising custom VLSI circuits or gate arrays, off-the-shelf semiconductors such as logic chips, transistors, or other discrete components. A module may also be implemented in programmable hardware devices such as field programmable gate arrays, programmable array logic, programmable logic devices or the like.

Modules may also be implemented in software for execution by various types of processors. An identified module of executable code may, for instance, comprise one or more physical or logical blocks of computer instructions which may, for instance, be organized as an object, procedure, or function. Nevertheless, the executables of an identified module need not be physically located together, but may comprise disparate instructions stored in different locations which, when joined logically together, comprise the module and achieve the stated purpose for the module.

Indeed, a module of executable code may be a single instruction, or many instructions, and may even be distributed over several different code segments, among different programs, and across several memory devices. Similarly, operational data may be identified and illustrated herein within modules, and may be embodied in any suitable form and organized within any suitable type of data structure. The operational data may be collected as a single data set, or may be distributed over different locations including over different storage devices, and may exist, at least partially, merely as electronic signals on a system or network.

Finally, the fact that the wording “an embodiment,” or the like, does not appear at the beginning of every sentence in the specification, such as is the practice of some practitioners, is merely a convenience for the reader's clarity. However, it is the intention of this application to incorporate by reference the phrasing “an embodiment,” and the like, at the beginning of every sentence herein where logically possible and appropriate.

As used herein, “comprising,” “including,” “containing,” “is,” “are,” “characterized by,” and grammatical equivalents thereof are inclusive or open-ended terms that do not exclude additional, unrecited elements or method steps. “Comprising” is to be interpreted as including the more restrictive terms “consisting of” and “consisting essentially of.”

Sleep disorders exist in various classes and magnitudes. Sleep disorders may be classified under three general headings: dyssomnias, parasomnias, and sleep disorders associated with mental, neurological, or other medical disorders. Dyssomnias produce either excessive sleepiness or difficulty in entering or maintaining sleep. These disorders include circadian rhythm disorders, insomnia, narcolepsy, motor disorders that disrupt sleep, and sleep apnea. Parasomnias are physical phenomena that occur during sleep. Some examples or parasomnias include nightmares, sleepwalking, sleep terrors, teeth grinding, and bed-wetting.

Sleep disorders associated with mental, neurological, or other medical disorders include sleep disorders that result from prenatal exposure to drugs, specifically stimulants such as methamphetamines and cocaine. It has been observed that the heart rate and the breathing rate of infants and children who have had prenatal exposure to such drugs may be rapid or erratic, thus preventing the infant or child from entering sleep or deep/REM sleep. As a result, such infants or children miss the sleep or deep/REM sleep that they need during their early years.

It has further been observed that the heart rate and/or the breathing rate of infants, children, and adults may be influenced by placing the person in close proximity with another person. A phenomenon has been observed wherein the infant, child, or adult will match the breathing rate and/or heart rate of the person near to whom they are placed. As a result, persons with sleep disorders may be helped into sleep or deep sleep by placing the person in close proximity with another person. Specifically it has been observed that infants and children who had prenatal exposure to drugs such as methamphetamines and/or cocaine may be helped into a sleep or deep sleep by placing the infant or child in close proximity with another person. For example, when such an infant or child is placed on the chest of an adult, the infant or child may match its heart rate and/or breathing rate to either the heart rate or breathing rate of the person on whose chest they are placed.

It is theorized, but not meant to be limiting, that the act (whether intentional or not) of matching a breathing rate and/or heart rate is a function of what is perceived. The perception may be, for example, by feeling, seeing, hearing, and the like. For example, when a human feels breathing movement, the human may attempt to match the felt breathing rate, or at least modify her breathing rate to more closely match that which is felt. It is one object of the present invention to provide a sleep aid that helps a human breathe by assisting her to modify her breathing rate to match detected movement. It is another object of the present invention to assist in the induction and/or maintenance of sleep by assisting a human modify her breathing rate and/or heart rate. It has been found that as the human matches her breathing rate to that which is sensed, the human may become more calm, less anxious, and the like, and may also be induced into sleep.

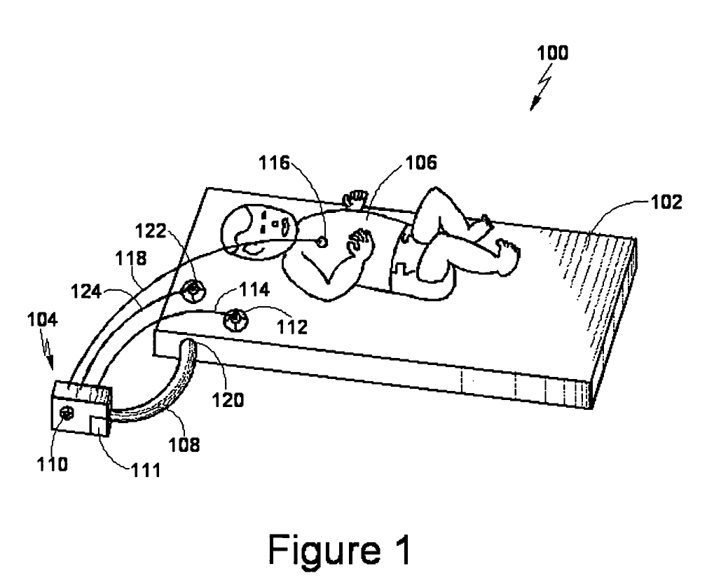

FIG. 1 illustrates a perspective view of the sleep aid according to one embodiment of the present invention. The illustrated sleep aid 100 includes a mattress 102. The illustrated sleep aid 100 may include a fluid pump 104 sealably connected to the mattress 102 by a fluid duct 120. The fluid pump 104 may be sealably connected to the mattress 102 by means of a conduit 108. The illustrated sleep aid 100 of the present invention may include a control module 111. The illustrated sleep aid 100 may include a heartbeat module 112 for simulating the sound of a human heartbeat. The heartbeat module 112 may be connected to the control module 111 by a conduit 114. The illustrated sleep aid 100 may include a detector module 116 for receiving a signal from a human 106 on the mattress 102. The detector module 116 may receive and transmit signals corresponding to a physiological event of the human 106 to the control module 111.

The mattress 102 may be configured to support the weight of the human 106 while the human 106 lay at least partially on the mattress 106. To this end, the mattress 102 may be filled with a fluid. The fluid may be any such as, for example, a gas, such as air, nitrogen, oxygen, helium, neon, argon, krypton, xenon, radon, or the like; a liquid, such as water, saline solution, glycerin, alcohol, glycol, or the like; a gel; or combinations thereof. Further, to support the weight, the mattress 102 may be substantially sealed so that the fluid does not substantially escape the mattress 102, except as allowed by the fluid pump 104 through the fluid duct 120.

The mattress 102 may be formed from a flexible material such that the modulate provided by the fluid pump 104 may be felt by the human 106. Some examples of materials that are substantially flexible such that the human 106 feels the modulates include, for example, plastics, such as polytetraflouroethylene, polyethylene, polypropylene, polybutylene, polystyrene, vinyl polymers, silicone polymers, polymers of more than one monomer, and the like; leather; woven textiles; non-woven textiles; and so forth.

The mattress 102 may include a fluid duct 120 through which the fluid may enter and/or exit the mattress 102 from/to the fluid pump 104. There may be a conduit 108 sealably connected to the fluid duct 120 and the fluid pump 104. The fluid pump 104 may be designed to force the fluid into and out of the mattress 102. Any fluid pump 104 capable of forcing the fluid into and out of the mattress 102 may be used. One such fluid pump is disclosed in U.S. Pat. No. 4,066,072 to Cummins, which is herein incorporated by a reference. The fluid pump 104 forces the fluid into and/or out of the mattress 102 at a modulate rate. The fluid pump 104 and/or the mattress 102 may be adjustable such that the amount of fluid modulated into and/or out of the mattress 102 is modifiable. The amount of fluid modulated into and/or out of the mattress 102 may correspond to the amount of motion of the mattress 102 as the fluid is modulated into and/or out of the mattress 102. In one embodiment, the amount of fluid modulated into and/or out of the mattress 102 may be controlled by the user. Any control known in the art may be used to control the amount of fluid modulated into and/or out of the mattress 102. The modulate may be controlled by the control module 111.

The control module 111 may be configured to control the modulate rate according to a modulate rate pattern. In one embodiment, the sleep aid 100 includes a timer module 110. The timer module 110 may function in conjunction with the control module 111, or may be a part of the control module 111. The timer module 110 may include a clock. The control module 111 may regulate the modulate rate in accordance with time kept by the timer module 110. In one embodiment, the control module 111 decreases the modulate rate stepwise over time. The modulate rate may be decreased to a predetermined minimum. In another embodiment, the control module 111 decreases the modulate rate continuously over time. Again, the modulate rate may be decreased to a predetermined minimum. The modulate rate may change according to a predetermined pattern.

The modulate rate pattern may follow a sine wave. The modulate rate pattern may mimic a breathing pattern of a sleeping human. The human sleeping pattern may be that of an adult. Prenatal infants become accustomed to the breathing pattern of their mother. It is theorized, but not meant to be limiting, that after birth, the infant may be affected by a breathing rate simulation that mimic's that of a mother. It is further theorized that the infant is able to control her own breathing rate to decrease and/or increase as the breathing rate of the mother increases or decreases, even after birth, as the infant hears and/or feels the breathing rate of the mother increase or decrease. Finally, it is theorized that any human is able to control his breathing rate to decrease and/or increase as a second breathing rate is heard and/or felt. In controlling the breathing rate, sleep patterns may also be controlled. Thus, by mimicking the breathing rate patterns of a sleeping human, the sleep aid of the present invention may provide a pattern which a human may follow to induce and/or maintain proper sleep patterns.

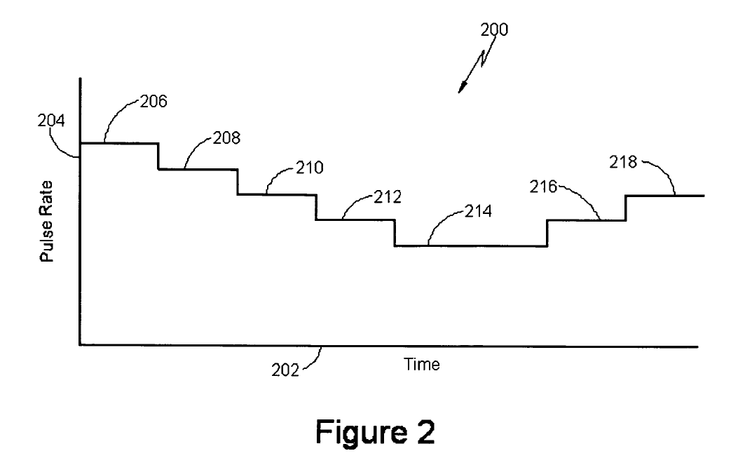

As can be seen in FIG. 2, the modulate rate 204 of the fluid pump 104 may decrease and/or increase stepwise over time 202. The modulate rate may begin at a level similar to that of wakefulness 206, and decrease stepwise over time 208, 210, and 212, until a minimum 214 is reached. These steps of decreasing may represent the first four stages of non-REM sleep and the stages of REM sleep, as breathing decreases and stabilizes. The modulate rate may then increase stepwise over time 216 and 218, representing a pattern of coming out or REM sleep. This pattern may continue for a predetermined amount of time.

According to one embodiment of the present invention, the control module 111 may be set such that it increases the modulate rate to a rate commensurate with that of wakefulness at a predetermined time, or after a predetermined amount of time. This embodiment may be useful for waking a human at a certain time or after a certain amount of sleep time.

The modulate rate may decrease and/or increase over predetermined time periods. The time periods may be determined in accordance with the normal sleep patterns of a human as described above. In one embodiment, the control module 111 may be set such that it decreases and increases the modulate rate stepwise over time according to a predetermined modulate rate corresponding to common breathing rates associated with stages of sleep. For example, the control module 111 may control the pump to start at a modulate rate of about 25 breaths per minute (corresponding to wakefulness). After a predetermined amount of time, the control module 111 may decrease the modulate rate to about 20 breaths per minute (corresponding to N-REM sleep). Further, the control module 111 may decrease the modulate rate in continuing steps, such as, for example, to about 18 breaths per minute (corresponding to N-REM and/or REM sleep), and even further steps for predetermined amounts of time. The control module 111 may then increase the modulate rate after a predetermined amount of tome to about 20 breaths per minute, and then to about 25 breaths per minute, for predetermined amounts of time. These last steps may correspond to the stages of sleep leading back to wakefulness. The following chart illustrates the modulate rate over time according to one embodiment of the present invention, and how the stages of sleep may fit into the modulate rate:

[image]

Alternatively to the above embodiments, the modulate rate may increase and/or decrease continuously instead of stepwise. The rate of increase or decrease may be constant or variable. That is, the modulate rate may decrease more quickly at first than when the rate approaches a set minimum.

In one embodiment, the sleep aid 100 is configured such that the fluid modulated into and/or out of the mattress 102 may simulate the sound of breathing. The sound of breathing may be made by a breathing module. The sound of breathing may also assist in the inducement and/or maintenance of sleep in a human 106. The breathing module may include a valve in the conduit 108 from the mattress 102 to the fluid pump 104.

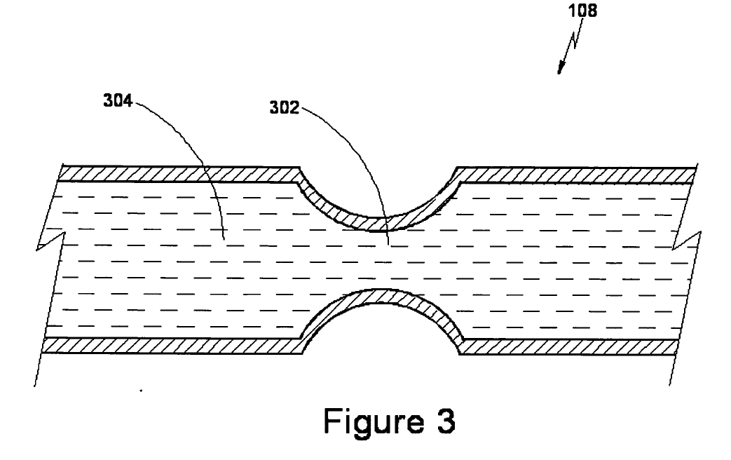

FIG. 3 illustrates a cross-sectional view of the conduit 108 according to one embodiment of the present invention where a valve 302 is placed in the conduit 108 to simulate the sound of breathing as a fluid 304 is modulated through the valve 302. U.S. Pat. No. 4,166,337 to Kosicki, which is herein incorporated by a reference, discloses a doll that makes a breathing sound. In one embodiment the breathing sound of the present invention follows the disclosure of the Kosicki patent in that the breathing sound is produced by forcing air through a curved conduit.

In another embodiment, the breathing module includes a speaker 122, which may be connected to the fluid pump 104 or the control module 111 by a conduit 124. The conduit 124 may be any capable of carrying a signal to the speaker 122. The speaker 122 may be placed on the mattress 102, on the fluid pump 104, or separate from either of these. The speaker 122 may reproduce the sound of breathing at a rate commensurate with the modulate rate. The volume of the breathing may be increased or decreased. There may be a volume control on the sleep aid 100 that controls the volume of the speaker 122. In this embodiment, the volume of the breathing sound may be increased or decreased. In another embodiment, the volume of the speaker 122 is controlled by the control module 111. Thus, the volume of the breathing sound may be increased and/or decreased according to a predetermined pattern, the length of time of use, and/or the time of day.

In one embodiment, the breathing module may be deactivated. In some situations, the breathing module may not induce and/or maintain sleep. The breathing module may be deactivated by blocking air flow through the valve that causes the sound. In one embodiment, the valve that causes the sound may be located in an alternative conduit from the fluid pump 104 to the mattress 102, and may contain a second valve that is closable, inhibiting air flow through the valve that produces the sound. If the breathing module includes a speaker, the sound may be deactivated by any means known in the art for deactivating a speaker, such as, for example, unplugging the speaker, turning a switch to deactivate the speaker, and so forth.

Turning again to FIG. 1, the sleep aid device 100 of the present invention may include a heartbeat module 112. The heartbeat module 112 may be configured to simulate the feel and/or sound of a human heartbeat. As described above, a prenatal infant may hear and/or feel the breathing of her mother. Likewise, the prenatal infant may hear and/or feel the beating of her mother's heart. As with breathing, human sleep may be affected by the sensing of a human heartbeat. Thus, it is theorized that when a human is exposed to a heartbeat pattern that simulates the heartbeat pattern of a sleeping human, the human may be induced to sleep or maintain sleep.

The heartbeat module 112 may simulate the sound of a human heartbeat. Any device known in the art capable of simulating the sound of a human heartbeat may be used. U.S. Pat. No. 4,066,072 to Cummins, discloses pressure actuated valves which simulate the sound of the valves of the human heart as they open and close. The heartbeat module 112 of the present invention may include valves as disclosed in the Cummins patent to simulate the sound of a human heartbeat. In a similar embodiment, the valves may be positioned such that the fluid flow from the valves as they open and close are directed such that the human may feel as well as hear the heartbeat simulation.

In another embodiment, the heartbeat module 112 may include a speaker. The speaker may be positioned on a surface of the mattress 102 such that the sound of the heartbeat may be transmitted through the mattress 102. Thus the sound of the heartbeat may be better heard when the ear is placed on the mattress 102. This embodiment may simulate sleeping on the torso of a human such as an infant sleeping on the torso of an adult. When one sleeps on the torso of another human, the breathing may be heard and/or felt, and the heartbeat may be heard. The sound of the heartbeat may be magnified when the ear is placed upon the torso. The speaker may be placed and/or configured such that the heartbeat may also be felt by a human 106 on the mattress 102. In one embodiment, the speaker is placed within the mattress 102 on a surface on which the human 106 sleeps, facing to direct vibrations toward the human 106. In this embodiment, the vibrations caused by the speaker may be heard by the human 106 as well as felt by the human 106 as the vibrations are transmitted to a surface of the mattress 102 on which the human 106 sleeps.

The heartbeat module 112 may be controlled by the control module 111. The control module 111 may be connected to the heartbeat module by means of a conduit 114. The conduit may be an electrical wire, a fluid flow conduit, or the like, depending on the type of heartbeat module 112 that is used. The rate of the simulated heartbeat may decrease and/or increase corresponding to the increase and/or decrease of the modulate rate of the fluid pump 104. The rate of the simulated heartbeat may decrease and/or increase stepwise. The rate of the simulated heartbeat may decrease and/or increase continuously. The rate of the simulated heartbeat may decrease to a predetermined minimum. The rate of the simulated heartbeat may follow a pattern. The pattern may mimic that of a sine wave. The pattern may follow a pattern of a sleeping human. The pattern may follow any of the patterns of the modulate rate, described herein.

The sleep aid 100 of the present invention may include a signal detector module module 116. The signal detector module module 116 may receive a signal corresponding to a physiological event from the human 106. The physiological event may be one associated with sleep such as, for example, breathing rate, modulate rate, muscle activity, and the like. The signal detector module module 116 may send signals corresponding to the physiological event to the control module 111. The signals may be sent by means of a conduit 118. The conduit 118 may be any configured to transmit the signal to the control module 111, such as, for example, a wire, fiber optics, and the like.

The control module 111 may receive the signals from the signal detector module module 116. The control module 111 may control the modulate rate and/or the rate of the heartbeat simulations in accordance with the signals received from the signal detector module module. Thus, the control module 111 may induce and/or maintain sleep of a human 106 by sensing the stage of sleep of the human 106. In one embodiment, the control module 111 causes the modulate rate and/or the rate of the heartbeat simulations to decrease only after a threshold of the received signal has been crossed. For example, the physiological event may be heartbeat. The threshold may be a heartbeat commensurate with the heartbeat of the first stage of non-REM sleep of the human 106. The control module 111 keeps the modulate rate and/or the rate of the heartbeat simulations constant until the signals received from the signal detector module module 116 are commensurate with the heartbeat of the first stage of non-REM sleep.

In one embodiment, the physiological activity includes the breathing rate of the human 106. The modulate rate of the sleep aid 100 may be modified to match the heartbeat of the human 106. The modulate rate of the sleep aid 100 may then be decreased to a predetermined level. The predetermined level may be a function of the breathing rate of the human 106. Once the breathing rate of the human 106 matches the modulate rate of the sleep aid 100, then the modulate rate of the sleep aid 100 may again be decreased to a predetermined level. This pattern may be continued until a predetermined minimum is reached.

In another embodiment, the control module 111 may continue to modify the modulate rate and/or rate of the heartbeat simulations during the various phases of sleep. For example, when the human 106 passes from REM sleep into non-REM sleep, the control module 111 may modify the modulate rate and/or rate of heartbeat simulations. The control module 111 may include an algorithm such that when the signals received from the signal detector module module 116 reach various thresholds after certain other thresholds have been crossed, that the modulate rate and/or rate of the heartbeat simulations are to be modified to certain levels. For example, when the human 106 passes into non-REM sleep for a second time, the control module 111 may modify the modulate rate and/or rate of heartbeat simulations to a predetermined level that is different from the predetermined level commensurate with the first time that the human 106 passes into non-REM sleep. Further, the duration of each time the human remains in REM sleep may be varied in accordance with a predetermined level.

The predetermined levels may consist of certain rates, or may be a function of the signals received from the signal detector module module 116. In the embodiment where the predetermined levels are functions of the signals received from the signal detector module module 116, the sleep aid may provide different thresholds, modulate rates, and/or rates of heartbeat simulations depending on the signals corresponding to a physiological event received from the signal detector module module 116. For example, if the signal corresponding to a physiological event received is a heartbeat, an adult human would provide different signals for different stages of sleep than would an infant. The control module 111 may adjust the thresholds and/or predetermined levels depending upon the heartbeat of the adult or infant using the sleep aid 100.

The control module 111 may receive signals from both the signal detector module module 116 and the timer module 110. Thus, the control module 111 may control the modulate rate and/or rate of heartbeat module to control the time lengths of each stage of sleep. The control module 111 may also control the modulate rate and/or rate of heartbeat module to control the timing of each stage of sleep, such that the human 106 enters sleep at a predetermined time, has each stage of sleep at a predetermined time, and/or wakes at a predetermined time.

The present invention also relates to a method of inducing and/or maintaining sleep in a human. The method may include the sleep aid of any of the embodiments herein described. The method may include the steps of placing a human on the mattress 102 of the sleep aid device, modulating fluid into and/or out of the mattress at a rate, and decreasing the rate. As described herein, it is believed that sleep in a human may be induced and/or maintained when breathing and/or heartbeat is sensed.

The method may include the step of providing the sound and/or feel of a heartbeat. The simulated heartbeat may repeat at a rate. The rate may be decreased. The rates of the simulated heartbeat and the fluid modulate may be controlled. They may be decreased. The method may include the step of increasing the rates. The method may include the step if decreasing and/or increasing the rates according to a predetermined pattern. The predetermined pattern may be any that is described herein. The pattern may follow a sleep pattern of a human.

The method may include the step of sensing a physiological event of a human. The sleep aid device may receive signals corresponding to a physiological event of a human. The sleep aid device may modify the rates of fluid modulate and/or heartbeat simulations as a result of the received signals that correspond to a physiological event. The physiological event may be any that is related to the sleeping patterns of the human, as discussed herein.

It is understood that the above-described preferred embodiments are only illustrative of the application of the principles of the present invention. The present invention may be embodied in other specific forms without departing from its spirit or essential characteristics. The described embodiment is to be considered in all respects only as illustrative and not restrictive. The scope of the invention is, therefore, indicated by the appended claims rather than by the foregoing description. All changes which come within the meaning and range of equivalency of the claims are to be embraced within their scope.

For example, the mattress of the present invention may be incorporated into another sleeping device such as a bassinet, cradle, crib, bed, another mattress, and the like. In one embodiment, the mattress of the present invention is included within a mattress upon which a human sleeps.

Other articles that may help induce and/or maintain sleep may also be incorporated into any embodiments of the present invention. One such article may include a temperature device. The temperature device may function to maintain and/or modify the temperature of the mattress. This may be accomplished by maintaining and/or modifying the temperature of the fluid that is modulated. In one embodiment, the temperature device may work to increase the temperature of the mattress or the fluid. In another embodiment, the temperature device may work to decrease the temperature of the mattress or the fluid. The temperature device may use any means known in the art for decreasing temperature, such as by evaporative cooling, compressive cooling, chemical reaction, and the like. In yet another embodiment, the mattress includes a temperature device that is a heating pad. The temperature device may be turned on and/or adjusted to increase and/or decrease the temperature, or it may be controlled by the control module 111. The temperature device may be controlled in response to a physiological event, a signal from which may be sent from the detector module to the control module 111. The physiological event may include any herein described, and/or the temperature of the human. Human temperature may change during the various stages of sleep. This embodiment may provide another aspect of inducing and/or maintaining sleep in that it may help the human keep a temperature pattern commensurate with the temperature pattern of a sleeping human.

It is also envisioned that the control module 111 may control the amount of fluid modulated into and/or out of the mattress. In this embodiment, the range of motion of the mattress may be controlled by the control module 111 by its control of the amount of fluid modulated into and/or out of the mattress.

It is expected that there could be numerous variations of the design of this invention. The control module 111 may be placed upon or incorporated into any part of the sleep aid. For example, the control module 111 may be incorporated into the mattress. Alternatively, the control module 111 may be separate from any other part of the sleep aid, and only connected to the sleep aid as needed to control the fluid pump 104 and/or heartbeat module, and/or receive signals from the detector module. The connection may be by any means known in the art such as, for example, electrical, electromagnetic (ultraviolet, infrared, radio, microwave, visible light, laser, and so forth), pneumatic, and so forth. Further, the fluid pump 104 may be part of the mattress.

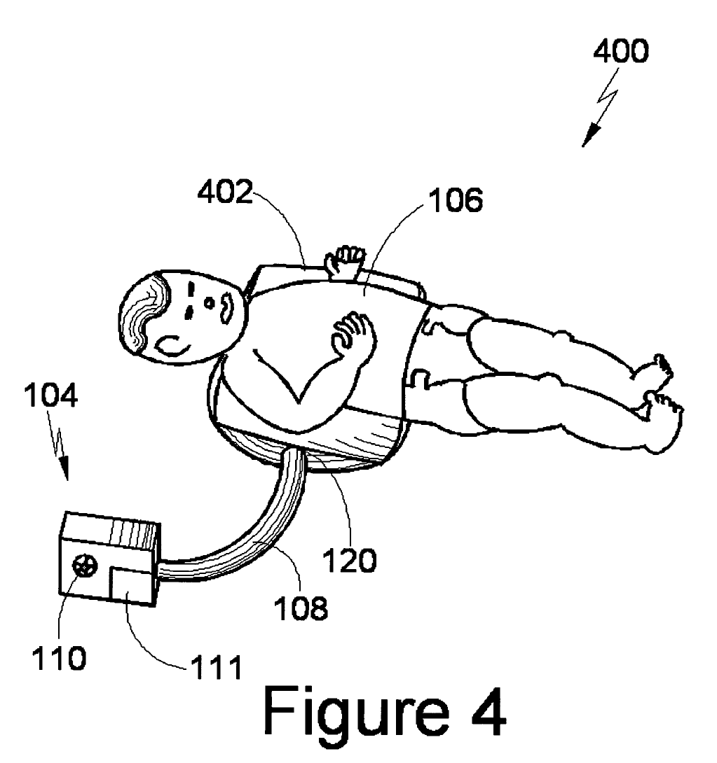

One particular embodiment of the present invention is illustrated in FIG. 4. The sleep aid of this embodiment includes a fluid pump 104 of any of the above embodiments, a mattress 402. The sleep aid may include a conduit 108 and a fluid duct 120. The mattress of this embodiment may be of a size sufficient to create a motion palpable by the human 106 as the fluid pump 104 forces the fluid into and/or out of the mattress 402. In one embodiment, the sleep aid may include any of the other components of any of the above embodiments. In another embodiment, the sleep aid may increase a thickness of the mattress 402 at least about 0.5 cm, or at least about 1 cm, or at least about 1.5 cm; and less than about 30 cm, or less than about 20 cm, or less than about 15 cm, when fluid is forced into the mattress 402. In yet another embodiment, the mattress 402 may have a thickness of at less than about 30 cm, or less than about 15 cm, or less than about 5 cm, when the fluid is forced out of the mattress. According to a further embodiment, the mattress 402 includes a thickness of about 3 cm when the fluid is forced out of the mattress 402. According to yet a further embodiment, the mattress 402 is of a size that the human 106 does not completely fit on the mattress 402. In still a further embodiment, the mattress 402 is of a size just larger than the torso of the human 106.

Thus, while the present invention has been fully described above with particularity and detail in connection with what is presently deemed to be the most practical and preferred embodiment of the invention, it will be apparent to those of ordinary skill in the art that numerous modifications, including, but not limited to, variations in size, materials, shape, form, function and manner of operation, assembly and use may be made, without departing from the principles and concepts of the invention as set forth in the claims.

What is claimed is:

1. A sleep aid for assisting the modification of a breathing rate in a human, comprising:
a substantially flexible mattress configured to receive a fluid;
a fluid pump on fluid communication with the substantially flexible mattress and configured to pump the fluid at a modulated rate; and
a control module functionally coupled to the fluid pump and configured to determine the modulated rate.

2. The sleep aid of claim 1, wherein the control module further comprises:
a detector module configured to detect a physiological event of a human; and
a transducer module functionally coupled to the detector module and configured to create a signal correlated to detection of the physiological event of the human by the detector.

3. The sleep aid of claim 1, wherein the control module further comprises a timer module.

4. The sleep aid of claim 1, wherein the control module causes the modulate rate to decrease stepwise over time to a predetermined modulate rate.

5. The sleep aid of claim 1, wherein the control module causes the modulate rate to decrease continuously over time to a predetermined modulate rate.

6. The sleep aid of claim 3, wherein the control module decreases the modulate rate as a function of time as determined by the timer module.

7. The sleep aid of claim 1, wherein the control module causes the modulate rate to decrease and increase in accordance with a pattern.

8. The sleep aid of claim 7, wherein the pattern corresponds to a breathing rate pattern of a sleeping adult human.

9. The sleep aid of claim 2, wherein the control module causes the modulate rate to decrease and increase corresponding with the signals received by the control module from the detector module.

10. The sleep aid of claim 1, further comprising a breathing module in fluid communication with the fluid pump that is configured to simulate the sound of breathing when a fluid passes therethrough.

11. The sleep aid of claim 1, further comprising a heartbeat module coupled to the control module and configured to simulate the sound of a heartbeat.

12. The sleep aid of claim 11, wherein the heartbeat module simulates the sound of a heartbeat at a rate controlled by the control module.

13. The sleep aid of claim 12, wherein the rate of the heartbeat increases and decreases with the modulate rate.

14. A method of assisting the modification of a breathing rate in a human, comprising the steps of:
placing the human on a substantially flexible mattress configured to receive a fluid;
modulating the fluid into the mattress at a first modulate rate; and
modulating the fluid into the mattress at a second modulate rate.

15. The method of claim 14, wherein the first and second modulate rates correspond to a sleep pattern of the human.

16. The method of claim 14, further comprising providing a sound simulating a heartbeat of a human.

17. The method of claim 14, wherein the second modulate rate is faster than the first modulate rate.

18. The method of claim 17, wherein the decreasing and increasing the modulate rate corresponds to a normal breathing rate pattern of a sleeping human.

19. The method of claim 14, further comprising the step of providing a sound which simulates breathing of a human.

20. The method of claim 19, wherein the sound simulating breathing of a human repeats at a rate corresponding to the rate of modulating the fluid into the mattress.
